(19) United States
(12) Patent Application Publication
(10) Pub. No.: US 2007/0245500 A1
(43) Pub. Date: Oct. 25, 2007

(54) Cosmetic composition comprising at least one cyanoacrylate monomer, at least one uncolored heat-stable organic particle and at least one liquid organic solvent and process for using it

(76) Inventors: Gaelle Brun, Paris (FR); Luc Gourlaouen, Asnieres (FR); Arnaud Bonnamy, Versailles (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,928

(22) Filed: Apr. 13, 2007

Related U.S. Application Data
(60) Provisional application No. 60/796,930, filed on May 3, 2006.

(30) Foreign Application Priority Data
Apr. 13, 2006 (FR) 06/03284

(51) Int. Cl. A61K 8/00

(52) U.S. Cl. 8/405

(57) ABSTRACT

The present disclosure relates to a cosmetic composition for treating keratin fibers; for instance human keratin fibers such as the hair, comprising at least one liquid organic solvent, at least one polymerizable cyanoacrylate monomer, and at least one uncolored heat-stable organic particle with a numerical mean primary size of less than 30 μm, with the exception of polytetrafluoroethylene particles.

This application claims benefit of U.S. Provisional Application No. 60/796,930, filed May 3, 2006, the contents of which are incorporated herein by reference. This application also claims benefit of priority under 35 U.S.C. § 119 to French Patent Application No. FR 0603284, filed Apr. 13, 2006, the contents of which are also incorporated herein by reference.

The present disclosure relates to a cosmetic composition for treating keratin fibers, for instance human keratin fibers such as the hair, comprising at least one polymerizable cyanoacrylate monomer, at least one uncolored heat-stable organic particle and at least one liquid organic solvent, to a use of this composition for treating the hair and also to a treatment process involving the composition as disclosed herein.

Hair is generally damaged and embrittled by the action of external atmospheric agents such as the light and bad weather, and by mechanical or chemical treatments such as blow-drying, combing, bleaching, permanent-waving and/or dyeing. As a result, the hair is often difficult to manage, for example, it may be difficult to disentangle or style. A head of hair, even of lush hair, can be difficult to maintain an attractive style due to the fact that the hair can lack vigor, volume and liveliness.

Thus, in order to overcome this, it is now common practice to use styling products that allow the hair to be conditioned by giving it body, mass or volume, for instance.

These styling products are generally cosmetic hair compositions comprising at least one polymer with high affinity for the hair, which usually have the function of forming a film on the hair surface in order to modify its surface properties, for instance, to condition it.

One drawback associated with the use of these hair compositions lies in the fact that the cosmetic effects imparted by such compositions have a tendency to disappear, even after the first shampoo wash.

In order to overcome this drawback, it may be envisaged to increase the remanence of the deposit of polymers by directly performing free-radical polymerization of certain monomers on the hair. However, the treatments used thus far tend to result in the degradation of the fiber in such a manner that the hair treated can be generally difficult to disentangle.

It is known practice, for example from French Patent FR 2 833 489, to use anionic-polymerizing electrophilic monomers directly at the surface of the hair in the presence of a nucleophilic agent such as hydroxide ions (OH⁻) contained in water at neutral pH. Thus, once applied to the hair, these monomers form a polymer leading to a coating.

However, the coating obtained using these compositions may not show satisfactory resistance in response to the various attacking factors to which the hair may be subjected.

Thus, there is a real need to find cosmetic compositions, for instance for conditioning the hair, that are remanent to shampooing and to external attack, while at the same time maintain good cosmetic properties, such as providing long-lasting body, mass or volume to the hair.

One aspect of the present disclosure relates to a cosmetic composition for treating keratin fibers, for instance human keratin fibers such as the hair, that comprises at least one liquid organic solvent, at least one polymerizable cyanoacrylate monomer and at least one uncolored heat-stable organic particle with a numerical mean primary size of less than 30 μm, with the exception of polytetrafluoroethylene particles.

Thus, the composition of the present disclosure, by combining at least one uncolored heat-stable organic particle with a numerical mean primary size of less than 30 μm, with at least one cyanoacrylate monomer, in at least one liquid organic solvent, makes it possible to give long-lasting body, mass or volume to keratin fibers such as the hair.

Thus, such a composition makes it possible to obtain a remenant coating that shows good resistance to the various attacking factors to which the hair may be subjected, for example to fatty substances, such as sebum, and to shampooing.

A coating can be obtained in the form of a smooth, uniform deposit that shows excellent adhesion to the hair.

Moreover, it has also been found that the hair remains perfectly individualized and can be styled without problem.

One aspect of the present disclosure relates to a cosmetic process using the cosmetic composition as disclosed herein.

Another aspect of the present disclosure relates to a method for using the cosmetic composition for treating keratin fibers.

A further aspect of the present disclosure relates to a multi-compartment kit comprising, at least one first compartment comprising a first cosmetic composition comprising at least one cyanoacrylate monomer and, at least one second compartment comprising a second cosmetic composition comprising at least one uncolored heat-stable organic particle as defined herein, and at least one liquid organic solvent.

Other subjects, characteristics, aspects and benefits of the present disclosure will emerge even more clearly upon reading the description and the examples that follow.

The at least one cyanoacrylate monomer present in the composition of the present disclosure may be chosen from the monomers of formula (I): 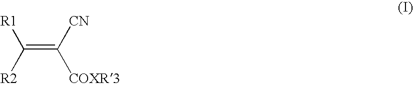
wherein:

X is chosen from NH, S and O,

R1 and R2, independently of each other, are chosen from sparingly and/or non-electron-withdrawing groups (sparingly and/or non-inductive-withdrawing) such as:
a hydrogen atom;
a saturated and unsaturated, linear, branched and cyclic hydrocarbon-based groups, containing from 1 to 20 for example, and further still from 1 to 10 carbon atoms, and optionally comprising at least one group chosen from nitrogen, oxygen and sulfur, and optionally substituted with at least one group chosen from —OR, —COOR, —COR, —SH, —SR and —OH, and halogens;
a modified or unmodified polyorganosiloxane residue; and
a polyoxyalkylene group;

R is chosen from saturated and unsaturated, linear, branched and cyclic hydrocarbon-based groups, for instance chosen from those comprising from 1 to 20, for example, from 1 to 10 carbon atoms, and optionally comprising at least one group chosen from nitrogen, oxygen and sulfur, and optionally substituted with at least one group chosen from —OR′, —COOR′, —COR′, —SH, —SR′ and —OH, halogens, and a polymer residue, this polymer possibly being obtained by free-radical polymerization, by polycondensation or by ring opening, where R— is chosen from C₁-C₁₀ alkyl groups.

The terms “electron-withdrawing” and/or “inductive-withdrawing group (−I)” as used herein, is generally understood to mean any group that is more electronegative than carbon. For example, reference may be made to the publication P. R. Wells, Prog. Phys. Org. Chem., Vol. 6, 111 (1968).

The term “sparingly and/or non-electron-withdrawing group” as used herein, is generally understood to mean any group whose electronegativity is less than or equal to that of carbon.

According to at least one embodiment, the alkenyl or alkynyl groups may contain from 2 to 20 carbon atoms and further, for example, from 2 to 10 carbon atoms.

As disclosed herein, the saturated and unsaturated, linear, branched and cyclic hydrocarbon-based groups may contain, for example, from 1 to 20 carbon atoms, and non-limiting mention may be made for instance of linear and branched alkyl, alkenyl and alkynyl groups, such as methyl, ethyl, n-butyl, tert-butyl, isobutyl, pentyl, hexyl, octyl, butenyl or butynyl; cycloalkyl and aromatic groups.

Examples of substituted hydrocarbon-based groups that may be mentioned include but are not limited to hydroxyalkyl and polyhaloalkyl groups.

Examples of unmodified polyorganosiloxanes that may be mentioned include but are not limited to polyalkylsiloxanes such as polydimethylsiloxanes, polyarylsiloxanes such as polyphenylsiloxanes, and polyarylalkylsiloxanes such as polymethylphenylsiloxanes.

Examples of the modified polyorganosiloxanes that may be mentioned include but are not limited to polydimethylsiloxanes containing polyoxyalkylene and/or siloxy and/or silanol and/or amine and/or imine and/or fluoroalkyl groups.

Examples of the polyoxyalkylene groups that may be mentioned include but are not limited to polyoxyethylene groups and polyoxypropylene groups such as those containing 1 to 200 oxyalkylene units.

Among the mono- or polyfluoroalkyl groups that may be mentioned include but are not limited to groups such as —(CH₂)ₙ—(CF₂)ₘ—CF₃ or —(CH₂)—(CF₂)_{m—CHF}₂ with n=1 to 20 and m=1 to 20.

The substituents R1 and R2 may optionally be substituted with a group having cosmetic activity. The cosmetic activities that may be used can be obtained from groups having coloring, antioxidant, UV-screening and conditioning functional groups, for example.

As examples of groups having a coloring function, non-limiting mention may be made, for example, of azo, quinone, methine, cyanomethine and triarylmethane groups.

As examples of groups having an antioxidant function, non-limiting mention may be made, for example, of groups of butylhydroxyanisole (BHA), butylhydroxytoluene (BHT) or vitamin E type.

As examples of groups having a UV-screening function, non-limiting mention: may be made, for example, of groups of the benzophenone, cinnamate, benzoate, benzylidenecamphor and dibenzoylmethane type.

As examples of groups having a conditioning function, non-limiting mention may be made, for example, of cationic groups and groups of fatty ester type.

According to at least one embodiment, as disclosed herein, R1 and R2 are hydrogen atoms,

R′3 is chosen from hydrogen and linear, branched and cyclic, saturated and unsaturated hydrocarbon-based groups that may contain from 1 to 20 carbon atoms, for instance, and further still from 1 to 10 carbon atoms, and optionally comprising at least one group chosen from nitrogen, oxygen and sulfur, and optionally substituted with at least one group chosen from —OR′, —COOR′, —COR′, —SH, —SR′ and —OH, and halogens, and a polymer residue that may be obtained by free-radical polymerization, by polycondensation or by ring opening, where R′ is chosen from C₁-C₁₀ alkyl groups.

For example, R′3 is chosen from C₁-C₁₀ saturated hydrocarbon-based groups.

According to at least one embodiment, X is O.

Compounds of formula (I) that may be mentioned include but are not limited to the monomers:
a) belonging to the family of polyfluoroalkyl 2-cyanoacrylates such as:
   the ester 2,2,3,3-tetrafluoropropyl 2-cyano-2-propenoate of formula (II): 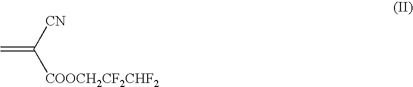
   or the ester 2,2,2-trifluoroethyl 2-cyano-2-propenoate of formula (Ill): 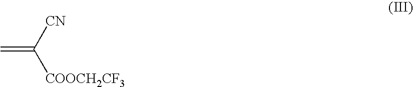
b) the alkyl or alkoxyalkyl 2-cyanoacrylates of formula (IV): 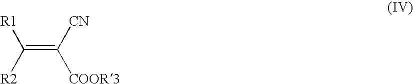
where R′3 is chosen from C₁-C₁₀, C₂-C₁₀ alkenyl and (C₁-C₄)alkoxy(C₁-C₁₀)alkyl radicals.

By way of example, non-limiting mention may be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, allyl 2-cyanoacrylate, methoxypropyl 2-cyanoacrylate and isoamyl cyanoacrylate.

As disclosed herein, at least one embodiment uses the monomers b). For instance, according to at least one embodiment, the at least one cyanoacrylate monomer is chosen from C₆C₁₀ alkyl cyanoacrylates.

According to at least one embodiment, the at least one cyanoacrylate monomer is chosen from the octyl cyanoacrylates of formula (V): 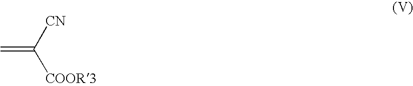
wherein: R—3=—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

The at least one cyanoacrylate monomer used in accordance with the present disclosure may be covalently bonded to at least one support such as polymers, oligomers and dendrimers. The polymer or the oligomer may be linear, branched, in comb form or in block form. The distribution of the monomers of the present disclosure over the polymeric, oligomeric and dendritic structure may be random, in an end position, and in the form of blocks.

The at least one cyanoacrylate monomer can be present in an amount ranging from 0.1% to 80% by weight, for example in an amount ranging from 0.2% to 60% by weight and even further, for example, in an amount ranging from 0.5% to 50% by weight relative to the total weight of the cosmetic composition.

In the context of the present disclosure, the at least one cyanoacrylate electrophilic monomer of formula (I) includes those monomers capable of undergoing anionic polymerization in the presence of at least one nucleophilic agent.

The term “anionic polymerization” as used herein, means the mechanism defined in the book “Advanced Organic Chemistry,” Third Edition by Jerry March, pages 151 to 161.

The nucleophilic agents capable of initiating the anionic polymerization are systems that are known per se, which are capable of generating a carbanion on contact with a nucleophilic agent, such as the hydroxide ions contained in water at neutral pH. The term “carbanion” as used herein, means the chemical species defined in “Advanced Organic Chemistry,” Third Edition, by Jerry March, page 141.

The at least one nucleophilic agent may be applied independent of the composition of the present disclosure. They may also be added to the composition of the present disclosure at the time of use.

The at least one nucleophilic agent can be chosen from molecular compounds, oligomers, dendrimers and polymers containing nucleophilic functional groups. In a non-limiting manner, the nucleophilic functional groups that may be mentioned include but are not limited to the following groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH^{-l , ArNH}₂, NH₃, I⁻, Br⁻, Cl⁻, RCOO⁻, SCN⁻, ROH, RSH, NCO⁻, CN⁻, NO₃⁻, ClO₄⁻and H₂, where Ph is a phenyl group; Ar is an aryl group, and R is a C₁-C₁₀ alkyl group.

The at least one cyanoacrylate monomer of formula (I) according to the present disclosure may be synthesized according to the known methods, as described in the art. For instance, the at least one cyanoacrylate monomer may be synthesized according to the teachings of U.S. Pat. Nos. 3,527,224; 3,591,767; 3,667,472; 3,995,641; 4,035,334 and 4,650,826.

As disclosed herein, the at least one particle used in the cosmetic composition includes uncolored heat-stable organic particles. For example, the particles can be polymeric and may be obtained from the polymerization of at least one monomer. The polymers constituting these organic particles may or may not be crosslinked.

For the purposes of the present disclosure, the term “heat-stable organic particles” means organic particles whose size do not vary by more than 20% when they are subjected to heating at 130° C. for two minutes.

For the purposes of the present disclosure, the term “uncolored organic particles” means organic particles which, when placed connectively on a support, i.e. when the particles are in contact with each other, show a difference in coloration with the white reference standard of the calorimeter that is less than or equal to 10, for instance less than 2.

The difference in coloration with the white reference standard of the colorimeter is evaluated by the color difference
DE=\sqrt{(Lᵢ*−L₀*)²+(aᵢ*−a₀*)²+(bᵢ*−b₀*)²}

Lᵢ*, aᵢ*, bᵢ* and L₀*, a₀*, b₀* being, respectively, the colorimetric coordinates in the La*b* system for the organic particles and for the white reference standard of the calorimeter.

In at least one embodiment, the organic particle(s) can have a numerical mean primary size that ranges from 0.1 to 30 μm, for instance from 0.2 to 20 μm and even further, for example from 0.5 to 15 μm.

For the purposes of the present disclosure, the term “primary particle size” means the maximum size that it is possible to measure between two diametrically opposite points on an individual particle.

The size of the organic particles may be determined by transmission electron microscopy or by measuring the specific surface area via the BET method or via laser granulometry.

The organic particle(s) used in the cosmetic composition may have various forms, for example a spherical, flake, needle or platelet form, and according to at least one embodiment, the organic particle is substantially spherical.

The organic particle(s) may be solid, hollow or porous.

When the organic particles are hollow, they comprise at least one continuous envelope (or one surface coat) and at least one cavity. According to at least one embodiment, the envelope for the particles is flexible to allow mechanical deformation. It generally comprises at least one polymer homopolymer or copolymer, formed from ethylenically unsaturated monomers.

The monomers that can be used include but are not limited to methacrylic or acrylic acid esters, such as methyl acrylate and methacrylate, vinylidene chloride, acrylonitrile, and styrene and derivatives thereof.

For purposes of the present disclosure, the term “porous particles” means particles with a structure comprising pores of variable number and size. The porosity associated with the size of the particles may be characterized quantitatively by measuring the specific surface area via the BET method.

According to at least one embodiment, the porous organic particles have a specific surface area of greater than or equal to 1 m²/g, such as greater than or equal to 2 m²/g and even further, for example, greater than or equal to 4 m²/g.

The specific surface area can be determined according to the BET (Brunauer-Emmet-Teller) method described in the Journal of the American Chemical Society, Vol. 60, page 309, February 1938, and corresponds to international standard ISO 5794/1 (appendix D). The specific surface area determined by the BET method corresponds to the total specific surface area, micropores included, of the organic particles under consideration.

The organic particle(s) used in the cosmetic composition according to the present disclosure may be chosen from polyamide powders, acrylic polymer powders, for example of polymethyl methacrylate, acrylic copolymer powders, such as of polymethyl methacrylate/ethylene glycol dimethacrylate, of polyallyl methacrylate/ethylene glycol dimethacrylate, of ethylene glycol dimethacrylate/lauryl methacrylate copolymer or of polyacrylate/alkylacrylate, polystyrene powders, polyethylene powders, such as polyethylene/acrylic acid, and silicone resin microbeads.

Non-limiting illustrations of organic particles according to the present disclosure that may be mentioned include, for example:

polyamide (Nylon®) powders, for example those sold under the names Orgasol®) 4000 and Orgasol®) 2002 UD NAT COS 204 by the company Atochem,

acrylic polymer powders, such as polymethyl methacrylate, for instance those sold under the name Covabead®) LH85 and Covabead® PMMA by the company Wackherr or those sold under the name Micropearl® MHB sold by the company Matsumoto,

acrylic copolymer powders, such as polymethyl methacrylate/ethylene glycol dimethacrylate, for instance those sold under the name Dow Corning 5640 Microsponge® Skin Oil Adsorber by the company Dow Corning, or those sold under the name Ganzpearl®) GMP-0820 by the company Ganz Chemical, of polyallyl methacrylate/ethylene glycol dimethacrylate, for instance those sold under the name Polypore® L200 or Polypore® E200 sold by the company Amcol, of ethylene glycol dimethacrylate/lauryl methacrylate copolymer, for instance those sold under the name Polytrap®) 6603 by the company Dow Corning, or of polyacrylate/ethylhexyl acrylate, for instance those sold under the name Techpolymer® ACX 806C by the company Sekisui,

polystyrene/divinylbenzene powders, for instance those sold under the name Techpolymer®) SBX8 by the company Sekisui,

polyethylene powders, for instance polyethylene/acrylic acid sold under the name Flobeads® by the company Sumitomo,

silicone resin microbeads, for instance those sold under the name Tospear® by the company Toshiba Silicone, in particular Tospearl® 240A and Tospearl® 120A.

According to at least one embodiment, the organic particles used in the composition in accordance with the present disclosure are chosen from polyamide powders and polymethyl methacrylate powders.

The organic particle(s) may be optionally surface-treated with a hydrophobic treatment agent.

Thus, the organic particles may be made hydrophobic by chemical coating or grafting with products such as:

silicones, for instance methicones or dimethicones,

amino acids, N-acylamino acids or salts thereof,

metal soaps, for instance aluminium dimyristate or the aluminium salt of hydrogenated tallow glutamate,

fluoro derivatives, for instance perfluoroalkylphosphates, perfluoroalkylsilanes, polyhexafluoropropylene oxides or polyorganosiloxanes comprising perfluoroalkyl perfluoropolyether groups,

lecithin or isopropyl triisostearyl titanate, and

fatty acids, for instance stearic acid.

The term “alkyl” as disclosed herein can be a linear, branched and cyclic alkyl group containing from 1 to 30 carbon atoms, for example those from 5 to 16 carbon atoms.

The N-acylamino acids may comprise an acyl group containing from 8 to 22 carbon atoms, for instance a 2-ethylhexanoyl, caproyl, lauroyl, myristoyl, palmitoyl, stearoyl or cocoyl group.

The salts of these compounds may be the aluminium, magnesium, calcium, zirconium, zinc, sodium or potassium salts.

The amino acid may be, for example, lysine, glutamic acid or alanine.

Examples that may be mentioned include but are not limited to polymethyl methacrylate microspheres coated with isopropyl triisostearyl titanate of size 2-15 μm sold by Kobo under the reference BPA-515.

The uncolored heat-stable organic particle(s) used in the cosmetic composition in accordance with the present disclosure may be present in an amount ranging from 0.1% to 15% by weight, for instance in an amount ranging from 0.2% to 10% by weight relative to the total weight of the cosmetic composition.

The term “organic solvent” is generally understood to mean an organic substance capable of dissolving another substance without chemically modifying it.

The liquid organic solvents can be chosen from compounds that are liquid at a temperature of 25° C. and at 105 Pa (760 mmHg) and are different from the cyanoacrylate monomers of the present disclosure.

The organic solvent can be chosen, for example, from:

aromatic alcohols such as benzyl alcohol; liquid fatty alcohols, and according to at least one embodiment, C₁₀-C₃₀; modified or unmodified polyols such as glycerol, glycol, propylene glycol, dipropylene glycol, butylene glycol or butyl diglycol; volatile silicones such as cyclopentasiloxane or cyclohexasiloxane; polydimethylsiloxanes optionally modified with alkyl and/or amine and/or imine and/or fluoroalkyl and/or carboxylic and/or betaine and/or quaternary ammonium functional groups, modified liquid polydimethylsiloxanes, mineral, organic and/or plant oils, alkanes such as C₅-C₁₀ alkanes; liquid fatty acids, liquid fatty esters for instance liquid fatty alcohol benzoates and salicylates.

As disclosed herein, the at least one organic solvent can be chosen from organic oils; silicones such as volatile silicones, amino or non-amino silicone gums or oils and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil or lemon oil, or alternatively organic compounds such as C₅-C₁₀ alkanes, acetone, methyl ethyl ketone, esters of liquid C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, esters of liquid C₁₀-C₃₀ fatty alcohols such as C₁₀-C₃₀ fatty alcohol benzoates, and mixtures thereof; polybutene oil, isononyl isononanoate, isostearyl malate, pentaerythrityl tetraisostearate, tridecyl trimellitate, and the mixture of cyclopentasiloxane (14.7% by weight)/polydimethylsiloxane dihydroxylated in the α and ω positions (85.3% by weight).

According to at least one embodiment, the at least one liquid organic solvent comprises at least one silicone such as liquid polydimethylsiloxanes and modified liquid polydimethylsiloxanes, their viscosity at 25° C. can range from 0.1 cSt to 1,000,000 cSt and further, for example, from cSt to 30,000 cSt.

Non-limiting mention can be made of the following oils:

the mixture of α,ω-dihydroxylated polydimethylsiloxane/cyclopentadimethylsiloxane (14.7/85.3) sold by Dow Corning under the name DC 1501 Fluid;

the mixture of α,ω-dihydroxylated polydimethylsiloxane/polydimethylsiloxane sold by Dow Corning under the name DC 1503 Fluid;

the mixture of dimethicone/cyclopentadimethylsiloxane sold by Dow Corning under the name DC 1411 Fluid or the product sold by Bayer under the name SF 1214;

the cyclopentadimethylsiloxane sold by Dow Corning under the name DC 245 Fluid;

and the respective mixtures of these oils.

Besides the at least one liquid organic solvent, the medium for the cosmetic compositions of the present disclosure may contain water.

According to at least one embodiment, the medium for the composition is an anhydrous medium, i.e. it contains less than 1% by weight of water relative to the total weight of the composition.

The at least one liquid organic solvent of the present disclosure is present in an amount ranging from 0.01% to 99%, for example, from 50% to 99% by weight relative to the total weight of the composition.

The cosmetic composition according to the present disclosure may further comprise at least one pigment.

As disclosed herein, the use of at least one pigment in the cosmetic composition makes it possible to obtain visible colorations for instance on dark hair, since the surface pigment masks the natural color of the fiber.

The composition in accordance with the present disclosure thus has the benefit of producing colorations that show good resistance to the various attacking factors to which the hair may be subjected, such as fatty substances or shampoos.

Furthermore, the cosmetic composition according to the present disclosure makes it possible to produce colorations that are visible and very chromatic on a keratin fiber, for instance on dark keratin fiber, without it being necessary to lighten or bleach the keratin fibers and, consequently, without causing the physical degradation of the keratin fibers.

For the purposes of the present disclosure, the term “pigment” means any organic and/or mineral species whose solubility in water is less than 0.01%, such as less than 0.0001% at 20° C., having an absorption that ranges from 350 to 700 nm and further, at maximum absorption.

According to at least one embodiment, the organic pigments have a difference in coloration with the white reference standard of the colorimeter of greater than 10.

According to another embodiment, the pigments used in the composition according to the present disclosure can be chosen for example, from organic and/or mineral pigments known in the art, including but not limited to those described in Kirk-Othmer's Encyclopedia of Chemical Technology and in Ullmann's Encyclopedia of Industrial Chemistry.

For instance, these pigments may be in the form of powder and pigmentary paste. They may be coated or uncoated.

The pigments in accordance with the present disclosure may be chosen, for example, from white or colored pigments, lakes, pigments with special effects such as nacres or flakes, and mixtures thereof.

By way of example, the mineral pigments that may be mentioned include but are not limited to titanium dioxide, which is or is not surface-treated, zirconium oxide or cerium oxide, iron oxide or chromium oxide, manganese violet, ultramarine blue, chromium hydrate and ferric blue. For example, the following mineral pigments may be used: Ta₂O₅, Ti₃O₅, Ti₂O₃, TiO, ZrO₂ as a mixture with TiO₂, ZrO₂, Nb₂O₅, CeO₂, ZnS.

By way of example, the organic pigments that may be mentioned include but are not limited to nitroso, nitro, azo, xanthene, quinoline, anthraquinone and phthalocyanin compounds, compounds of metallic complex type, and isoindolinone, isoindoline, quinacridone, perinone, perylene, diketopyrrolopyrrole, thioindigo, dioxazine, triphenylmethane and quinophthalone compounds.

As disclosed herein, the organic pigments can be chosen from carmine, carbon black, aniline black, azo yellow, quinacridone, phthalocyanin blue, sorghum red, the blue pigments codified in the Color Index under the references Cl 42090, 69800, 69825, 73000, 74100, 74160, the yellow pigments codified in the Color Index under the references Cl 11680, 11710, 15985, 19140, 20040, 21100, 21108, 47000, 47005, the green pigments codified in the Color Index under the references Cl 61565, 61570, 74260, the orange pigments codified in the Color Index under the references Cl 11725,15510, 45370, 71105, the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in French Patent No. FR 2 679 771.

According to at least one embodiment of the present disclosure, pigmentary pastes of organic pigment can be used, such as the products sold by the company Hoechst under the names:
Jaune Cosmenyl IOG: Pigment Yellow 3 (Cl 11710);
Jaune Cosmenyl G: Pigment Yellow 1 (Cl 11680);
Orange Cosmenyl GR: Pigment Orange 43 (Cl 71105);
Rouge Cosmenyl R: Pigment Red 4 (Cl 12085);
Carmin Cosmenyl FB: Pigment Red 5 (Cl 12490);
Violet Cosmenyl RL: Pigment Violet 23 (Cl 51319);
Bleu Cosmenyl A2R: Pigment Blue 15.1 (Cl 74160);
Vert Cosmenyl GG: Pigment Green 7 (Cl 74260); and
Noir Cosmenyl R: Pigment Black 7 (Cl 77266).

The pigments in accordance with the present disclosure may also be in the form of composite pigments as described in European Patent No. EP 1 184 426. These composite pigments may be composed, for example, of particles comprising a mineral core, at least one binder for fixing the organic pigments to the core, and at least one organic pigment at least partially covering the core.

As disclosed herein, the term “lakes” means dyes adsorbed onto insoluble particles, the assembly thus obtained remaining insoluble during use. The mineral substrates onto which the dyes are adsorbed are, for example, alumina, silica, calcium sodium borosilicate or calcium aluminium borosilicate, and aluminium. Among the organic dyes that may be mentioned is cochineal carmine.

Examples of lakes that may be mentioned include but are not limited to the products known under the following names: D & C Red 21 (Cl 45 380), D & C Orange 5 (Cl 45 370), D & C Red 27 (Cl 45 410), D & C Orange 10 (Cl 45 425), D & C Red 3 (Cl 45 430), D & C Red 7 (Cl 15 850:1), D & C Red 4 (Cl 15 510), D & C Red 33 (Cl 17 200), D & C Yellow 5 (Cl 19 140), D & C Yellow 6 (Cl 15 985), D & C Green (Cl 61 570), D & C Yellow 1 0 (Cl 77 002), D & C Green 3 (Cl 42 053), and D & C Blue 1 (Cl 42 090).

As disclosed herein, the term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). They are consequently in contrast with colored pigments, which afford a standard opaque, semi-transparent or transparent uniform shade.

By way of example, pigments with special effects that may be mentioned include white nacreous pigments such as mica coated with titanium or with bismuth oxychloride, colored nacreous pigments such as mica coated with titanium and with iron oxides, mica coated with titanium and further, for example with ferric blue or with chromium oxide, mica coated with titanium and with an organic pigment as defined above, and also nacreous pigments based on bismuth oxychloride. Nacreous pigments that may be mentioned include but are not limited to the Cellini nacre sold by Engelhard (Mica-TiO2-lake), the Prestige nacre sold by Eckart (Mica-TiO2), the Prestige Bronze nacre sold by Eckart (Mica-Fe2O3) and the Colorona nacre sold by Merck (Mica-TiO2-Fe2O3).

Non-limiting mention may also be made of pigments with an interference effect not bound to a substrate, for instance liquid crystals (Helicones HC from Wacker), holographic interference flakes (Geometric Pigments or Spectra f/x from Spectratek). The pigments with special effects may also comprise fluorescent pigments, whether they are substances that are fluorescent in daylight or that produce ultraviolet fluorescence, phosphorescent pigments, photochromic pigments, thermochromic pigments and quantum dots, for example those sold by the company Quantum Dots Corporation.

Quantum dots are luminescent semiconductive nanoparticles capable of emitting, under light excitation, radiation with a wavelength in the range from 400 nm to 700 nm. These nanoparticles are known in the literature. For instance, they may be manufactured according to the processes described, for example, in U.S. Pat. Nos. 6,225,198; and 5,990,479, in the publications cited therein and also in the following publications: Dabboussi B. O. et al “(CdSe)ZnS core-shell quantum dots: synthesis and characterisation of a size series of highly luminescent nanocristallites” Journal of Physical Chemistry B, Vol 101, 1997, pp. 9463-9475 and Peng, Xiaogang et al, “Epitaxial Growth of highly Luminescent CdSe/CdS core/shell nanocrystals with photostability and electronic accessibility” Journal of the American Chemical Society, Vol.119, No. 30, pp. 7019-7029.

The variety of pigments that maybe used according to the present disclosure make it possible to obtain a rich palette of colors, and also provide optical effects such as metallic or interference effects.

According to at least one embodiment, the pigments are colored pigments. As disclosed herein, the term “colored pigments” means pigments other than white pigments.

The size of the pigment that is useful within the context of the present disclosure can generally range from 10 nm to 200 μm, for example from 20 nm to 80 μm and even further, for example from 30 nm to 50 μm.

The pigments may be coated with organic or mineral compounds.

According to at least one embodiment, the organic agent with which the pigments are treated can be deposited on the pigments by evaporation of solvent, chemical reaction between the molecules of the surface agent or creation of a covalent bond between the surface agent and the pigments or fillers.

For the purposes of the present disclosure, the surface treatment is such that a surface-treated pigment conserves the intrinsic pigmenting properties that it had before treatment.

The surface treatment may thus be performed, for example, by chemical reaction of a surface agent with the surface of the pigments and creation of a covalent bond between the surface agent and the pigments or the fillers. For instance, this method is described in U. S. Pat. No. 4,578,266.

According to at least one embodiment as disclosed herein, an organic agent covalently bonded to the pigments can be used.

According to at least one embodiment, the agent for the surface treatment may be present in an amount ranging from 0.1% to 50% by weight, such as from 0.5% to 30% by weight and even further, for example, from 1% to 10% by weight relative to the total weight of the surface-treated pigments or fillers.

According to at least one embodiment of the present disclosure, the surface treatments of the pigments can be chosen from the following treatments:
a PEG-silicone treatment, for instance the AQ surface treatment sold by LCW;
a chitosan treatment, for instance the CTS surface treatment sold by LCW;
a triethoxycaprylylsilane treatment, for instance the AS surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a lauroyllysine treatment, for instance the LL surface treatment sold by LCW;
a lauroyllysine dimethicone treatment, for instance the LU/SI surface treatment sold by LCW;
a magnesium myristate treatment, for instance the MM surface treatment sold by LCW;
an aluminium dimyristate treatment, for instance the MI surface treatment sold by Miyoshi;
a perfluoropolymethylisopropyl ether treatment, for instance the FHC surface treatment sold by LCW;
an isostearyl sebacate treatment, for instance the HS surface treatment sold by Miyoshi;
a disodium stearoyl glutamate treatment, for instance the NAI surface treatment sold by Miyoshi;
a dimethicone/disodium stearoyl glutamate treatment, for instance the SA/NAI surface treatment sold by Miyoshi;
a perfluoroalkyl phosphate, for instance the PF surface treatment sold by Daito;
an acrylate/dimethicone copolymer and perfluoroalkyl phosphate treatment, for instance the FSA treatment sold by Daito;
a polymethylhydrogen siloxane/perfluoroalkyl phosphate treatment, for instance the IS01 surface treatment sold by Daito;
a lauroyllysine/aluminium tristearate treatment, for instance the LL-AISt surface treatment sold by Daito;
an octyltriethylsilane treatment, for instance the OTS surface treatment sold by Daito;
an octyltriethylsilane/perfluoroalkyl phosphate treatment, for instance the FOTS surface treatment sold by,Daito;
an acrylate/dimethicone copolymer treatment, for instance the ASC surface treatment sold by Daito;
an isopropyl titanium triisostearate treatment, for instance the ITT surface treatment sold by Daito;
a microcrystalline cellulose and carboxymethylcellulose treatment, for instance the AC surface treatment sold by Daito;
a cellulose treatment, for instance the C2surface treatment sold by Daito;
an acrylate copolymer treatment, for instance the APD surface treatment sold by Daito; and
a perfluoroalkyl phosphate/isopropyl titanium triisostearate treatment, for instance the PF+ITT surface treatment sold by Daito.

The at least one pigment is generally present in the composition, according to the present disclosure, in an amount ranging from 0.05% to 50% by weight, for instance from 0.1% to 35% by weight relative to the total weight of the composition.

The medium of the composition of the present disclosure may also be in the form of an emulsion and/or may be encapsulated, the at least one electrophilic monomer being maintained in an anhydrous medium until the time of use. When the medium is an emulsion, this emulsion may comprise, for example, a dispersed or continuous phase that may comprise water, C1-C4 aliphatic alcohols or mixtures thereof and an anhydrous organic phase comprising the at least one monomer. In the case of capsules or microcapsules, the capsule may contain the monomer in an anhydrous medium and may be dispersed in an anhydrous medium as defined above, water or C1-C4 aliphatic alcohols, or mixtures thereof.

According to at least one embodiment, the composition may further comprise at least one polymerization inhibitor, and further, for example, anionic and/or free-radical polymerization inhibitors, in order to enhance the stability of the composition over time. In a non-limiting manner, the following polymerization inhibitors that may be mentioned include but are not limited to: sulfur dioxide, nitric oxide, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole or hydroxyanisole, pyrogallol and derivatives thereof, p-methoxyphenol, hydroxybutyltoluene, alkyl sulfates, alkyl sulfites, alkyl sulfones, alkyl sulfoxides, alkyl sulfides, mercaptans and 3-sulfonene, and mixtures thereof. According to at least one embodiment, the alkyl groups may contain 1 to 6 carbon atoms.

As disclosed herein, mineral or organic acids may also be used as the at least one polymerization inhibitor.

Thus, the cosmetic composition according to the present disclosure may also comprise at least one mineral and/or organic acid, this acid containing at least one group chosen from carboxylic and sulfonic group, with a pKa in the range from 0 to 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid, trifluoroacetic acid, octanoic acid, heptanoic acid and hexanoic acid.

According to at least one embodiment, acetic acid is used.

As disclosed herein, the concentration of the at least one polymerization inhibitor in the cosmetic composition may range from 10 ppm to 30% by weight, for example from 10 ppm to 15% by weight relative to the total weight of the composition.

To modify the anionic polymerization kinetics, it is also possible to increase the nucleophilicity of the fiber via chemical conversion of the keratin material.

Examples that may be mentioned include but are not limited to the reduction of the disulfide bridges of which keratin is partly composed, into thiols, before applying the composition of the present disclosure. In a non-exhaustive manner, as reducing agents for the disulfide bridges of which keratin is partially composed, mention may be made of the following compounds:
anhydrous sodium thiosulfate,
powdered sodium metabisulfite,
thiourea,
ammonium sulfite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulfoxylate,
isooctyl thioglycolate,
dI-cysteine, and
monoethanolamine thioglycolate.

According to at least one embodiment, the composition of the present disclosure may also comprise at least one polymer with no reactivity on the at least one cyanoacrylate monomer and which is capable of increasing the viscosity of the composition. Increasing the viscosity can make it possible to reduce the rate of polymerization of the at least one cyanoacrylate monomer. In order to do this, it is possible to add to the composition of the present disclosure, in a non-exhaustive manner, polymethyl methacrylate (PMMA) or cyanoacrylate-based copolymers as described in patent U.S. Pat. No. 6,224,622.

As disclosed herein, the compositions may also comprise at least one adjuvant usually used in cosmetics, for instance reducing agents, fatty substances, plasticizers, softeners, antifoams, moisturizers, pigments, clays, mineral fillers, UV-screening agents, mineral colloids, peptizers, solubilizing agents, fragrances, preserving agents, anionic, cationic, nonionic or amphoteric surfactants, fixing or non-fixing polymers, polyols, proteins, vitamins, direct dyes or oxidation dyes, nacreous agents, propellent gases, mineral or organic thickeners such as benzylidene sorbitol and N-acylamino acids, oxyethylenated or non-oxyethylenated waxes, paraffins, and C₁₀-C₃₀ fatty amides such as lauric diethanolamide.

The compositions may be in various galenical forms such as a lotion, an aerosol mousse, a hair conditioner or a shampoo, a gel or a wax. The compositions may be packaged, for example, in a pump-dispenser bottle or an aerosol spray container. The compositions of the present disclosure, after application, may be rinsed out or left in.

When the composition is packaged in an aerosol container, it may comprise at least one propellant. The at least one propellant can comprise the compressed or liquefied gases usually used for the preparation of aerosol compositions. For instance, air, carbon dioxide, compressed nitrogen or a soluble gas such as dimethyl ether, halogenated hydrocarbons (for example, fluorinated hydrocarbons) or non-halogenated hydrocarbons (butane, propane or isobutane), and mixtures thereof, can be used.

According to the process of the present disclosure, the composition as disclosed herein is applied to the keratin fibers, for instance human keratin fibers such as the hair, in the presence of at least one nucleophilic agent.

According to at least one embodiment of the process of the present disclosure, the at least one nucleophilic agent is capable of initiating the polymerization of the at least one electrophilic monomer and may be applied beforehand to the keratin fibers. The at least one nucleophilic agent may be used pure, as a solution, in the form of an emulsion or may be encapsulated; It may also be added to the cosmetic composition as disclosed herein at the time of use just before application to the keratin fibers.

According to at least one embodiment, the at least one nucleophilic agent is water. This water may be supplied, for example, by premoistening the keratin fibers. It may also be added directly to the composition before application.

According to at least one embodiment, it is possible to modify the polymerization kinetics by premoistening the fiber with an aqueous solution whose pH has been adjusted using a base, an acid or an acid/base mixture. The acid and/or the base may be mineral and/or organic.

According to at least one embodiment, the treatment process comprises a step of applying the uncolored heat-stable organic particles to human keratin fibers, such as the hair, followed by a step of applying at least one cyanoacrylate monomer and at least one liquid organic solvent to the keratin fibers.

According to another embodiment, the process for treating the keratin fibers may be a hair dyeing process, which may be performed in several steps: a first step that comprises applying a composition containing the pigment(s) to the fibers and a second step that comprises applying the composition according to the present disclosure comprising, inter alia, the at least one cyanoacrylate monomer, the uncolored heat-stable organic particle and the at least one liquid organic solvent, the at least one nucleophilic agent can be present in the composition containing the pigment or in a separate composition.

According to this embodiment, the cosmetic composition comprising the at least one pigment is an aqueous dispersion of pigments, which allows humidification of the fiber and initiation of the polymerization when the composition comprising the at least one cyanoacrylate monomer, the organic particle and the at least one liquid organic solvent is applied.

According to another embodiment of the present disclosure, the process comprises applying the liquid organic solvent, the cyanoacrylate monomer, the organic particle and optionally the pigments from the same composition.

Another process of the present disclosure may comprise additional intermediate or final steps such as applying a cosmetic product, a rinsing step and/or a drying step. The drying may be performed under a hood, with a hairdryer and/or with a smoothing iron. According to at least one embodiment, the application of the compositions in accordance with the present disclosure may be followed by rinsing.

It is also possible to perform multiple applications of the composition of the present disclosure in order to obtain a superposition of coats to achieve specific properties of the deposit in terms of chemical nature, mechanical strength, thickness, appearance or feel.

The present disclosure also relates to the use of a cosmetic composition as described above for treating the hair.

When the cosmetic composition according to the present disclosure does not comprise pigments, the cosmetic composition may be used for conditioning the hair.

Thus, when the cosmetic composition according to the present disclosure comprises both the organic particle and a pigment, the said composition may be used for coloring the hair and optionally for conditioning it.

A further aspect of the present disclosure is also a multi-compartment kit, comprising at least one first compartment comprising a composition comprising at least one polymerizable cyanoacrylate monomer as defined above and optionally at least one anionic and/or free-radical polymerization inhibitor, and at least one second compartment comprising a second composition comprising the at least one liquid organic solvent and at least one uncolored heat-stable organic particle as defined above, one of the two or both the compositions possibly comprising at least one cosmetic adjuvant as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without being limiting in nature.

### EXAMPLES

### Example 1

A styling composition (A) according to the present disclosure was prepared from the following compounds:

Composition A

| | | |
|---|---|---|
| α,ω-Dihydroxylated polydimethylsiloxane/ | 45 | g |
| cyclopentadimethylsiloxane (14.7/85.3) sold by Dow Corning | | |
| under the name DC 1501 Fluid | | |
| Cyclopentadimethylsiloxane sold by Dow Corning under | 41.75 | g |
| the name DC245 Fluid | | |
| Hollow polymethyl methacrylate microspheres | 3 | g |
| (specific surface area 50 m²/g) sold under | | |
| the name Covabead LH85 by LCW | | |
| Methylheptyl cyanoacrylate from Chemence | 10 | g |
| Acetic acid | 0.25 | g |

0.3 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

Composition B

A styling composition (B) according to the present disclosure was prepared from the following compounds:

| | | |
|---|---|---|
| DC 1501 Fluid | 45 | g |
| DC 245 Fluid | 41.75 | g |
| Polyamide 12 microspheres (specific surface area 9.5 m²/g) | 3 | g |
| sold under the name Orgasol 2002 UD NAT COS 204 by | | |
| Arkema | | |
| Methylheptyl cyanoacrylate from Chemence | 10 | g |
| Acetic acid | 0.25 | g |

0.3 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

Composition C

A styling composition (C) according to the present disclosure was prepared from the following compounds:

| | | |
|---|---|---|
| DC 1501 Fluid | 45 | g |
| DC 245 Fluid | 41.75 | g |
| Microspheres of the copolymer of polymethyl methacrylate | 3 | g |
| and of ethylene glycol dimethacrylate, sold under the name | | |
| Techpolymer MB30X-5 by Sekisui | | |
| Methylheptyl cyanoacrylate from Chemence | 10 | g |
| Acetic acid | 0.25 | g |

0.3 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

Composition D

A styling composition (D) according to the present disclosure was prepared from the following compounds:

| | | |
|---|---|---|
| DC 1501 Fluid | 45 | g |
| DC 245 Fluid | 41.75 | g |
| Polystyrene/divinylbenzene microspheres sold under the name | 3 | g |
| Techpolymer SBX-8 by Sekisui | | |
| Methylheptyl cyanoacrylate from Chemence | 10 | g |
| Acetic acid | 0.25 | g |

0.3 g of the composition was applied to a lock of 1 g of clean, wet hair. After a leave-on time of 15 minutes, the lock was dried with a hairdryer for 2 minutes. The resultant lock had hairs that were individualized with a pleasant, remanent texture.

### Example 2

A cosmetic composition (E) according to the present disclosure was prepared from the following compounds:

Composition E

| | | |
|---|---|---|
| DC 1501 Fluid | 40 | g |
| DC 245 Fluid | 36.75 | g |
| Mica nacre coated with brown iron oxide, sold by Eckart | 10 | g |
| under the name Prestige Bronze | | |
| Hollow polymethyl methacrylate microspheres (specific | 3 | g |
| surface area 50 m²/g) sold under the name Covabead LH85 by | | |
| LCW | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

Composition F

A cosmetic composition (F) according to the present disclosure were prepared from the following compounds:

| | | |
|---|---|---|
| DC 1501 Fluid | 40 | g |
| DC 245 Fluid | 36.75 | g |
| Polyamide 12 microspheres (specific surface area 9.5 m²/g) | 3 | g |
| sold under the name Orgasol 2002 UD NAT COS 204 | | |
| sold by Arkema | | |
| Mica nacre coated with brown iron oxide, sold by Eckart | 10 | g |
| under the name Prestige Bronze | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

Composition G

A cosmetic composition (G) according to the present disclosure was prepared from the following compounds:

| | | |
|---|---|---|
| DC 1501 Fluid | 40 | g |
| DC 245 Fluid | 36.75 | g |
| Microspheres of the copolymer of polymethyl methacrylate | 3 | g |
| and of ethylene glycol dimethacrylate, sold under the name | | |
| Techpolymer MB30X-5 by Sekisui | | |
| Mica nacre coated with brown iron oxide, sold by Eckart | 10 | g |
| under the name Prestige Bronze | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

Composition H

A cosmetic composition (H) according to the present disclosure was prepared from the following compounds:

| | | |
|---|---|---|
| DC 1501 Fluid | 40 | g |
| DC 245 Fluid | 36.75 | g |
| Polystyrene/divinylbenzene microspheres sold under the name | 3 | g |
| Techpolymer SBX-8 by Sekisui | | |
| Mica nacre coated with brown iron oxide, sold by Eckart | 10 | g |
| under the name Prestige Bronze | | |
| Methylheptyl cyanoacrylate from Chemence | 10 | g |
| Acetic acid | 0.25 | g |

0.5 g of the composition was applied to a lock of 1 g of clean, wet hair. After a leave-on time of 15 minutes, the lock was dried under a hairdryer for 2 minutes. The resultant lock had hairs that were individualized with a pleasant, remanent texture.

What is claimed is::

1. A cosmetic composition for treating keratin fibers, comprising:
at least one liquid organic solvent;
at least one polymerizable cyanoacrylate monomer; and
at least one uncolored heat-stable organic particle with a numerical mean primary size of less than 30 μm, with the exception of polytetrafluoroethylene particles.

2. The cosmetic composition according to claim 1, wherein the at least one cyanoacrylate monomer is chosen from the monomers of formula (I): 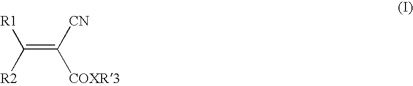
wherein:
X is chosen from NH, S and O,
R1 and R2 are chosen from, independently of each other, sparingly and non-electron-withdrawing groups;
R is chosen from saturated and unsaturated, linear, branched and cyclic hydrocarbon-based groups, and optionally comprising at least one group chosen from nitrogen, oxygen and sulfur, and optionally substituted with at least one group chosen from —OR′, —COOR′, —COR′, —SH, —SR′ and —OH, halogens, and a polymer residue, this polymer possibly being obtained by free-radical polymerization, by polycondensation or by ring opening, and where R′ is chosen from C₁-C₁₀ alkyl group,
R′3 is chosen from a hydrogen atom and linear, branched and cyclic, saturated and unsaturated hydrocarbon-based groups, optionally comprising at least one group chosen from nitrogen, oxygen and sulfur, and optionally substituted with at least one group chosen from —OR′, —COOR′, —COR′, —SH, —SR′ and —OH, and halogens, and a polymer residue that may be obtained by free-radical polymerization, by polycondensation or by ring opening, where R′ is chosen from C₁-C₁₀ alkyl groups.

3. The cosmetic composition according to claim 2, wherein
R1 and R2 are chosen from, independently of each other:
a hydrogen atom;
saturated and unsaturated, linear, branched and cyclic hydrocarbon-based groups, optionally comprising at least one group chosen from nitrogen, oxygen and sulfur, and optionally substituted with at least one group chosen from —OR, —COOR, —COR, —SH, —SR and —OH, and halogens;
a modified or unmodified polyorganosiloxane residue; and
a polyoxyalkylene group.

4. The cosmetic composition according to claim 2, wherein the at least one cyanoacrylate monomer is chosen from the monomers of formula (IV): 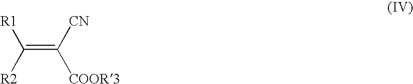
wherein R′3 is chosen from C₁-C₁₀ alkyl, C₂-C₁₀ alkenyl or (C₁-C₄) alkoxy(C₁-C₁₀)alkyl radicals.

5. The cosmetic composition according to claim 4, wherein the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate.

6. The cosmetic composition according to claim 5, wherein the at least one cyanoacrylate monomer is chosen from C₆-C₁₀ alkyl cyanoacrylates.

7. The cosmetic composition according to claim 6, wherein the at least one cyanoacrylate monomer is chosen from the octyl cyanoacrylate monomers of formula (V): 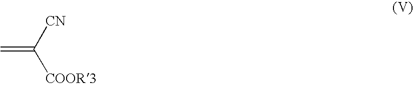
wherein: R′3=—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

8. The cosmetic composition according to claim 1, wherein the at least one cyanoacrylate monomer is covalently bonded to at least one support chosen from polymers, oligomers and dendrimers.

9. The cosmetic composition according to claim 1, wherein the at least one cyanoacrylate monomer is present in an amount ranging from 0.1% to 80% by weight relative to the total weight of the composition.

10. The cosmetic composition according to claim 9, wherein the at least one cyanoacrylate monomer is present in an amount ranging from 0.5% to 50% by weight relative to the total weight of the composition.

11. The cosmetic composition according to claim 1, wherein the at least one uncolored heat-stable organic particle has a numerical mean primary size ranging from 0.1 to 30 μm.

12. The cosmetic composition according to claim 11, wherein the at least one uncolored heat-stable organic particle has a numerical mean primary size ranging from 0.5 to 15 μm.

13. The cosmetic composition according to claim 1, wherein the at least one organic particle is chosen from hollow and porous organic particles.

14. The cosmetic composition according to claim 13, wherein the at least one organic particle is porous.

15. The cosmetic composition according to claim 14, wherein the at least one porous organic particle has a specific surface area of greater than or equal to 1 m²/g.

16. The cosmetic composition according to claim 15, wherein the at least one porous organic particle has a specific surface area of greater than or equal to 4 m²/g.

17. The cosmetic composition according to claim 1, wherein the at least one organic particle is chosen from polyamide powders, acrylic polymer powders, acrylic copolymer powders, polystyrene powders, polyethylene powders and silicone resin microbeads.

18. The cosmetic composition according to claim 17, wherein the at least one organic particle is chosen from polyamide powders and polymethyl methacrylate powders.

19. The cosmetic composition according to claim 1, wherein the at least one uncolored heat-stable organic particle is present in an amount ranging from 0.1% to 20% by weight relative to the total weight of the cosmetic composition.

20. The cosmetic composition according to claim 19, wherein the at least one uncolored heat-stable organic particle is present in an amount ranging from 0.5% to 10% by weight relative to the total weight of the cosmetic composition.

21. The cosmetic composition according to claim 1, wherein the at least one liquid organic solvent is chosen from aromatic alcohols; liquid fatty alcohols; modified and unmodified polyols; volatile silicones optionally modified with alkyl and/or amine and/or imine and/or fluoroalkyl and/or carboxylic and/or betaine and/or quaternary ammonium functional groups, modified liquid polydimethylsiloxanes, mineral, organic and/or plant oils, alkanes; liquid fatty acids, liquid fatty esters, and fatty alkyl benzoates and/or salicylates.

22. The cosmetic composition according to claim 21, wherein the at least one liquid organic solvent comprises a mixture of α,ω-dihydroxylated polydimethylsiloxane/cyclopentadimethylsiloxane and of cyclopentadimethylsiloxane.

23. The cosmetic composition according to claim 21, wherein the at least one organic solvent is present in an amount ranging from 0.01% to 99% by weight relative to the total weight of the cosmetic composition.

24. The cosmetic composition according to claim 23, wherein the at least one organic solvent is present in an amount ranging from 50% to 99% by weight relative to the total weight of the cosmetic composition.

25. The cosmetic composition according to claim 1, further comprising at least one pigment.

26. The cosmetic composition according to claim 25, wherein the at least one pigment is present in an amount ranging from 0.1% to 50% by weight relative to the total weight of the composition.

27. The cosmetic composition according to claim 26, wherein the at least one pigment is present in an amount ranging from 1% to 10% by weight relative to the total weight of the composition.

28. The cosmetic composition according to claim 1, further comprising at least one polymerization inhibitor.

29. The cosmetic composition according to claim 28, wherein the at least one polymerization inhibitor is a mineral or organic acid chosen from phosphoric acid, hydrochloric acid, nitric acid, benzenesulfonic acid or toluenesulfonic acid, sulfuric acid, carbonic acid, hydrofluoric acid, acetic acid, formic acid, propionic acid, benzoic acid, mono-, di- or trichloroacetic acid, salicylic acid, trifluoroacetic acid, octanoic acid, heptanoic acid and hexanoic acid.

30. The cosmetic composition according to claim 29, wherein the acid is acetic acid.

31. The-cosmetic composition according to claim 28, wherein the at least one polymerization inhibitor is present in an amount ranging from 10 ppm to 30% by weight relative to the total weight of the cosmetic composition.

32. The cosmetic composition according to claim 31, wherein the at least one polymerization inhibitor is present in an amount ranging from 10 ppm to 15% by weight relative to the total weight of the cosmetic composition.

33. The cosmetic composition according to claim 1, wherein the cosmetic composition is anhydrous.

34. The cosmetic composition according to claim 1, further comprising at least one nucleophilic agent.

35. The cosmetic composition according to claim 34, wherein the at least one nucleophilic agent is water.

36. A cosmetic process for treating keratin fibers, comprising:
applying a cosmetic composition to the keratin fibers in the presence of at least one nucleophilic agent, wherein the cosmetic composition comprises:
at least one liquid organic solvent;
at least one polymerizable cyanoacrylate monomer; and
at least one uncolored heat-stable organic particle with a numerical mean primary size of less than 30 μm, with the exception of polytetrafluoroethylene particles.

37. The cosmetic process according to claim 36, wherein the cosmetic composition comprises at least one nucleophilic agent.

38. The cosmetic process according to claim 36, wherein the at least one nucleophilic agent is water.

39. A cosmetic process comprising:
applying a first cosmetic composition to the keratin fibers in the presence of at least one nucleophilic agent, wherein the first cosmetic composition comprises at least one pigment; and
applying a second cosmetic composition to the keratin fibers, wherein the second cosmetic composition comprises:
at least one liquid organic solvent;
at least one polymerizable cyanoacrylate monomer; and at least one uncolored heat-stable organic particle with a numerical mean primary size of less than 30 pm, with the exception of polytetrafluoroethylene particles.

40. The hair dyeing process according to claim 39, wherein the first cosmetic composition further comprises the at least one nucleophilic agent.

41. The hair dyeing process according to claim 39, wherein the at least one nucleophilic agent is in a separate composition.

42. A method for providing a remnant coating on human keratin fibers comprising applying a cosmetic composition on human keratin fibers, wherein the cosmetic composition comprises:
at least one liquid organic solvent;
at least one polymerizable cyanoacrylate monomer; and
at least one uncolored heat-stable organic particle with a numerical mean primary size of less than 30 μm, with the exception of polytetrafluoroethylene particles.

43. A multi-compartment kit, comprising:
at least one first compartment comprising a first composition comprising at least one polymerizable cyanoacrylate monomer and optionally at least one anionic and/or free-radical polymerization inhibitor; and
at least one second compartment comprising a second composition comprising at least one liquid organic solvent and at least one uncolored heat-stable organic particle.
